(19) United States
(12) Patent Application Publication
(10) Pub. No.: US 2019/0328249 A1
(43) Pub. Date: Oct. 31, 2019

(54) INTRACRANIAL PRESSURE MEASURING DEVICE

(71) Applicants: THE ASAN FOUNDATION, Seoul (KR); UNIVERSITY OF ULSAN FOUNDATION FOR INDUSTRY COOPERATION, Ulsan (KR)

(72) Inventors: Deok Hee LEE, Seoul (KR); Jung Cheol PARK, Seoul (KR); Seung Joo LEE, Sejong-si (KR); Sung Hoon KIM, Seoul (KR); Sang Beom JEON, Seoul (KR); Seon Moon HWANG, Bucheon-si (KR); Tae Il KIM, Seoul (KR); Chang Mo HWANG, Seoul (KR); Jun Young MAENG, Seoul (KR)

(21) Appl. No.: 16/467,581

(22) PCT Filed: Dec. 7, 2017
(86) PCT No.: PCT/KR2017/014314

§ 371 Date: Jun. 7, 2019

(30) Foreign Application Priority Data
Dec. 7, 2016 (KR) 10-2016-0166195

(51) Int. Cl. A61B 5/03; A61B 5/00

(52) U.S. Cl. CPC A61B 5/031; A61B 5/0215; A61B 5/6876

(57) ABSTRACT

An intracranial pressure measuring device includes: a sensor unit which measures a pressure at a predetermined position in a cerebral blood vessel of a subject; a wire unit which is connected to the sensor unit and inserted into the subject to position the sensor unit at the position; and an intracranial pressure measuring unit which measures an intracranial pressure based on the measured pressure.

### BACKGROUND

### Field

The present application relates to an intracranial pressure measuring device.

### Description of the Related Art

In general, as the number of elderly people at home increases due to the overall aging of the society, there is gradually increasing interest in welfare and care for the elderly people, such that it has started to approach the health care for the elderly people in new ways from various angles, and measurement of a blood pressure, which is the basis of a medical examination, attracts the attention.

When the cranial suture is fully engaged as the growth is completed, the brain is completely surrounded by a hard cranium. The limited intracranial space is occupied by cerebral parenchyma (about 1400 g, 80%), cerebrospinal fluid (150 mL, 10%), and blood (150 mL, 10%). As a volume of any one of the three parts is increased, volumes of the other parts are decreased to compensate for the increase in pressure (Monro-Kelli hypothesis).

However, if this compensatory action is not enough, the intracranial pressure is eventually increased. The increase in intracranial pressure means an increase in resistance in terms of hemodynamics in the brain, such that a cerebral perfusion pressure is decreased, which causes ischemia in a severe case.

The monitoring of the intracranial pressure (ICP) is very important to detect abnormal brain states such as intracranial hemorrhage, hydrocephalus, or brain tumor. The ICP monitoring has been performed so far through an invasive method that causes many disadvantages including the risk of infection, hemorrhage, or brain herniation.

FIG. 1 is a view for explaining various examples of intracranial pressure measuring devices in the related art. Referring to FIG. 1, the intracranial pressure measuring device may measure the intracranial pressure through ventriculostomy. The ventriculostomy may mean a procedure of accessing a cerebral ventricle of a brain by surgically penetrating a cranium, a dura mater, and the brain with a long needle, a catheter, or the like. The ventriculostomy may be used for a patient with subarachnoid hemorrhage, but there may be a limitation in that the ventriculostomy may be used only in a case in which the cerebral ventricle is large in order to measure the intracranial pressure by inserting the catheter into the cerebral ventricle.

In addition, the intracranial pressure measuring device may measure a pressure in the cerebral parenchyma by forming a hole in the cranium (skull) and inserting a needling sensor. The intracranial pressure may be measured by using a subdural bolt or a subdural catheter.

However, there is a problem in that there is a likelihood that an adverse effect such as cerebral hemorrhage may occur because of the invasive process of inserting the catheter directly into the cerebral parenchyma through these methods.

The background art of the present application is disclosed in Korean Patent Application Laid-Open No. 10-2007-0106004 (published on Oct. 31, 2007).

### SUMMARY

The present application has been made in an effort to provide an intracranial pressure measuring device that measures an intracranial pressure in a minimally invasive manner by applying a vascular access method used for angiography.

The present application has also been made in an effort to provide an intracranial pressure measuring device capable of more accurately measuring an intracranial pressure by being positioned maximally close to an inner wall in a cerebral blood vessel by using the intracranial pressure measuring device having a stent shape.

The present application has also been made in an effort to provide an intracranial pressure measuring device capable of measuring a pressure in a cerebral blood vessel by obstructing a blood flow by using the intracranial pressure measuring device having a distal coil unit and a proximal coil unit.

The present application has also been made in an effort to provide an intracranial pressure measuring device that measures a pressure in a cerebral blood vessel by applying micro electro-mechanical systems (MEMS).

However, technical problems to be solved by the exemplary embodiment of the present application are not limited to the aforementioned technical problem, and other technical problems may be present.

As a technical solution for achieving the above-mentioned technical objects, an intracranial pressure measuring device according to one exemplary embodiment of the present application includes: a sensor unit which measures a pressure at a predetermined position in a cerebral blood vessel of a subject; a wire unit which is connected to the sensor unit and inserted into the subject to position the sensor unit at the position; and an intracranial pressure measuring unit which measures an intracranial pressure based on the measured pressure.

According to one exemplary embodiment of the present application, the intracranial pressure measuring device may further include a stent which has a cylindrical structure opened at both ends thereof, is inserted into the cerebral blood vessel, and has one region therein in which the sensor unit is disposed, in which the wire unit may be connected to the stent.

According to one exemplary embodiment of the present application, the sensor unit may move to be close to an inner wall of the cerebral blood vessel as the stent autonomously expands in the cerebral blood vessel.

According to one exemplary embodiment of the present application, the sensor unit may move to be close to the inner wall in a direction of cerebral parenchyma.

According to one exemplary embodiment of the present application, the intracranial pressure measuring device may further include a marker for identifying a position or a direction of the sensor unit.

According to one exemplary embodiment of the present application, a position of the marker may be determined based on a position or a direction in which the sensor unit is disposed in the stent.

According to one exemplary embodiment of the present application, the cerebral blood vessel in which the stent in which the sensor unit is disposed is positioned may be a cerebral vein.

According to one exemplary embodiment of the present application, the intracranial pressure measuring device may further include: a first coil unit positioned at one side in a direction in which the sensor unit is inserted into the subject; and a second coil unit positioned at one side in a direction opposite to the direction, in which the wire unit is connected to at least one of the first coil unit, the sensor unit, and the second coil unit.

According to one exemplary embodiment of the present application, the first coil unit and the second coil unit may obstruct a blood flow toward the sensor unit.

According to one exemplary embodiment of the present application, the cerebral blood vessel in which the first coil unit and the second coil unit are positioned may be a cerebral artery.

According to one exemplary embodiment of the present application, the first coil unit and the second coil unit may obstruct a blood flow toward the sensor unit as the first coil unit and the second coil unit autonomously expand in the cerebral blood vessel.

According to one exemplary embodiment of the present application, the sensor unit may include an antenna for transmitting a measurement result, the antenna may transmit the measurement result to the intracranial pressure measuring unit in a wireless manner, and the intracranial pressure may be measured based on the measurement result.

According to one exemplary embodiment of the present application, the wire unit may include a separate information transmitting line in a wire and may transmit the measurement result in respect to the pressure in the cerebral blood vessel measured by the sensor unit to the intracranial pressure measuring unit.

According to one exemplary embodiment of the present application, the intracranial pressure measuring device may further include a transmitter for transmitting the measurement result in respect to the pressure in the cerebral blood vessel, in which the transmitter is positioned in a blood vessel of an arm and a neck and transmits the measurement result in respect to the pressure in the cerebral blood vessel to an external monitoring device and the intracranial pressure measuring unit.

According to one exemplary embodiment of the present application, the wire unit may position the sensor unit at a predetermined position in the cerebral blood vessel of the subject, and the wire unit may be separated and withdrawn from the cerebral blood vessel.

The above-mentioned technical solution is just illustrative but should not be interpreted as being intended to limit the present application. In addition to the above-mentioned exemplary embodiment, additional exemplary embodiments may be present in the drawings and the detailed description of the invention.

According to the above-mentioned technical solution of the present application, it is possible to provide the intracranial pressure measuring device that measures an intracranial pressure in a minimally invasive manner by applying a vascular access method used for angiography.

In addition, according to the above-mentioned technical solution of the present application, it is possible to provide the intracranial pressure measuring device capable of more accurately measuring an intracranial pressure by being positioned maximally close to an inner wall in a cerebral blood vessel by using the intracranial pressure measuring device having the stent shape.

In addition, according to the above-mentioned technical solution of the present application, it is possible to provide the intracranial pressure measuring device capable of measuring a pressure in a cerebral blood vessel by obstructing a blood flow by using the intracranial pressure measuring device having the distal coil unit and the proximal coil unit.

In addition, according to the above-mentioned technical solution of the present application, it is possible to provide the intracranial pressure measuring device that measures a pressure in a cerebral blood vessel by applying micro electro-mechanical systems (MEMS).

In addition, according to the above-mentioned technical solution of the present application, it is possible to provide the intracranial pressure measuring device capable of continuously monitoring an intracranial pressure by positioning the sensor unit in a cerebral blood vessel.

### BRIEF DESCRIPTION OF THE DRAWINGS

The above and other aspects, features and other advantages of the present disclosure will be more clearly understood from the following detailed description taken in conjunction with the accompanying drawings, in which:

FIG. 1 is a view for explaining various examples of intracranial pressure measuring devices in the related art,

FIG. 2 is a configuration view of an intracranial pressure measuring device according to an exemplary embodiment of the present application,

FIG. 3 is a view illustrating a first exemplary embodiment of the intracranial pressure measuring device according to the exemplary embodiment of the present application,

FIG. 4 is a view illustrating a second exemplary embodiment of the intracranial pressure measuring device according to the exemplary embodiment of the present application; and

FIG. 5 is a view schematically illustrating an example of a process of measuring an intracranial pressure by applying the first exemplary embodiment of the intracranial pressure measuring device according to the exemplary embodiment of the present application.

### DETAILED DESCRIPTION OF THE EMBODIMENT

Hereinafter, exemplary embodiments of the present application will be described in detail with reference to the accompanying drawings so that those with ordinary skill in the art to which the present application pertains may easily carry out the exemplary embodiments. However, the present application may be implemented in various different ways and is not limited to the exemplary embodiments described herein. A part irrelevant to the description will be omitted in the drawings in order to clearly describe the present application, and similar constituent elements will be designated by similar reference numerals throughout the specification.

Throughout the specification of the present application, when one constituent element is referred to as being “connected to” another constituent element, one constituent element can be “directly connected to” the other constituent element, and one constituent element can also be “electrically connected to” the other element with other elements therebetween.

Throughout the specification, when one member is disposed “on”, “at an upper side of”, “at an upper end of”, “below”, “at a lower side of”, or “at a lower end of” another member in the present specification of the present application, this includes not only a case where the one member is brought into contact with another member, but also a case where still another member is present between the two members.

Throughout the specification of the present application, unless explicitly described to the contrary, the word “comprise” or “include” and variations, such as “comprises”, “comprising”, “includes” or “including”, will be understood to imply the inclusion of stated constituent elements, not the exclusion of any other constituent elements.

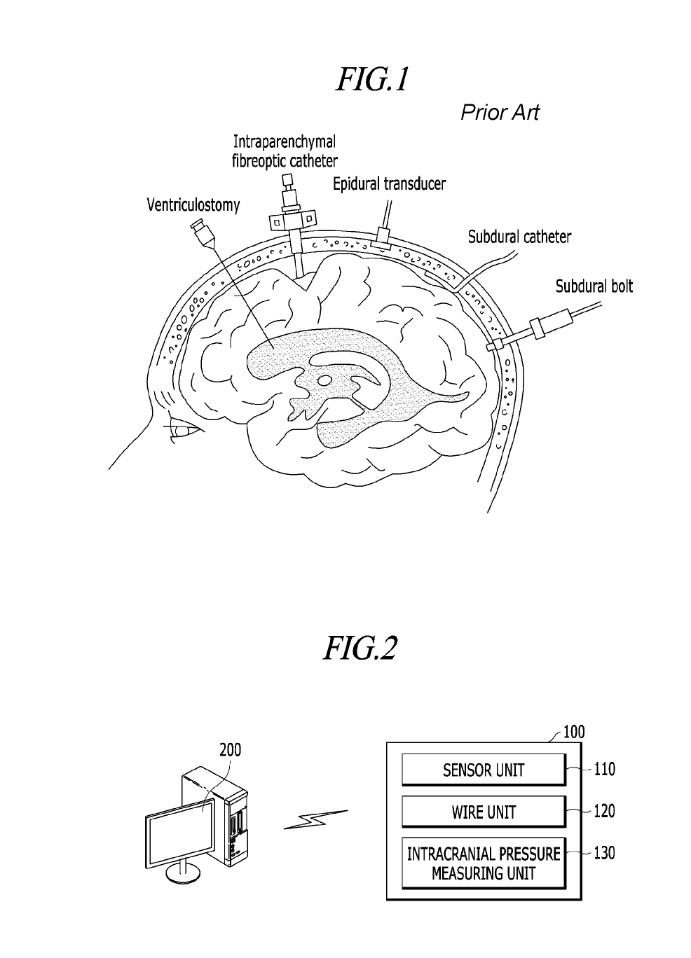

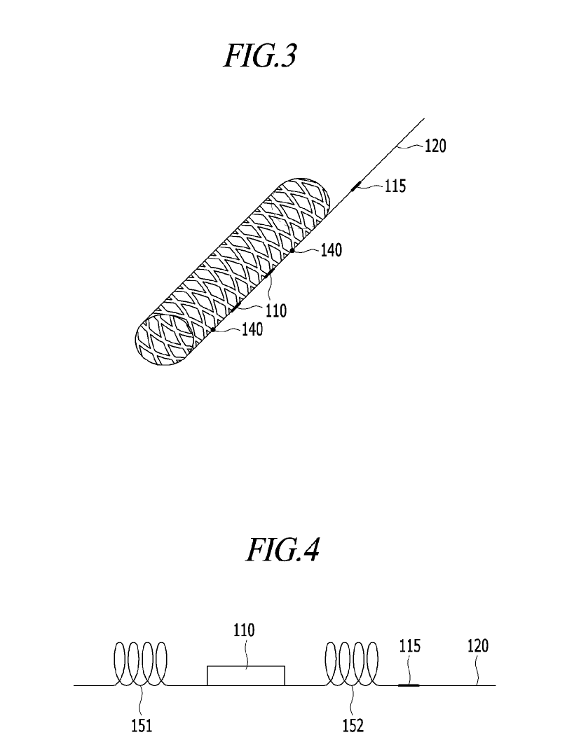

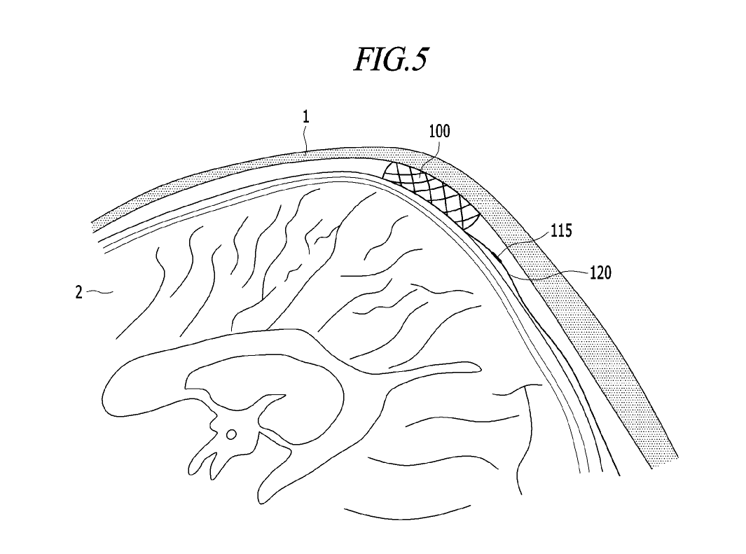

FIG. 2 is a configuration view of an intracranial pressure measuring device according to an exemplary embodiment of the present application, FIG. 3 is a view illustrating an exemplary embodiment of the intracranial pressure measuring device according to the exemplary embodiment of the present application, and FIG. 4 is a view illustrating another exemplary embodiment of the intracranial pressure measuring device according to the exemplary embodiment of the present application.

According to an exemplary embodiment of the present application, an intracranial pressure measuring device 100 may measure a pressure at a predetermined position in a cerebral blood vessel of a subject and may measure an intracranial pressure based on the measured pressure. The intracranial pressure measuring device 100 may measure the intracranial pressure in a minimally invasive manner by applying a vascular access method used for angiography. The intracranial pressure measuring device 100 may include a telemetric function that remotely controls a monitoring device connected to the intracranial pressure measuring device 100 in a wireless manner, such that the intracranial pressure measuring device 100 may continuously monitor the intracranial pressure at a remote place.

In addition, the intracranial pressure measuring device 100 is a device for measuring the intracranial pressure through the cerebral blood vessel, and the intracranial pressure measuring device 100 may be positioned in a cerebral artery, a cerebral vein or a blood vessel of a dura mater and may measure the intracranial pressure. The intracranial pressure measuring device 100 may be shaped to access the cerebral blood vessel in a minimally invasive manner, and the intracranial pressure measuring device 100 may be positioned maximally close to an inner wall in the cerebral blood vessel in order to more accurately measure a pressure in the cerebral blood vessel. The intracranial pressure measuring device 100 has a marker and may determine whether the intracranial pressure measuring device 100 is positioned at a predetermined position in a cerebral blood vessel of a subject by recognizing a position of the marker from the outside through a radioactive ray device and an image device.

According to one exemplary embodiment of the present application, the intracranial pressure may be measured based on the Monroe-Kelly doctrine. The Monroe-Kelly doctrine may mean that a cranium and components (blood, cerebrospinal fluid, and cerebral tissue) of the cranium create a volume equilibrium state, and an increase in volume of one of the components of the brain needs to be compensated by a decrease in volume of the other components. In this case, a main buffer liquid in respect to the increased volume includes the amount of cerebrospinal fluid (CSF) and the amount of blood.

V_{intracranial vault}=V_{brain}+V_{blood}+V_{csf} [Equation 1]

As shown in Equation 1, an intracranial space (intracranial vault) is occupied by cerebral tissue, blood, and cerebrospinal fluid, and volumes of the three components may be almost constant. Therefore, a decrease in volume of one component needs to be compensated by an increase in volume of the other components. For example, when a volume of the cerebral tissue (brain) is decreased, a volume of the blood in the brain is increased to a volume of the blood that corresponds to the decreased volume of the cerebral tissue, such that the overall volume of the brain may be constantly maintained. That is, when a volume of any one of the three components is increased, volumes of the other components are decreased to compensate for the increase in pressure (Monro-Kelli hypothesis). It is possible to measure the intracranial pressure by using the concept of the Monro-Kelli hypothesis. As an example, the intracranial pressure measuring device 100 may measure a change in blood pressure in the brain by using the concept in which at least one of a change in intracranial pressure, a change in cerebral tissue, and a change in cerebrospinal fluid is closely related to a change in blood in the brain, and the intracranial pressure measuring device 100 may determine the intracranial pressure based on the change in blood pressure in the brain, but the present application is not limited thereto.

Referring to FIG. 2, the intracranial pressure measuring device 100 may include, but not limited to, a sensor unit 110, a wire unit 120, and an intracranial pressure measuring unit 130. As an example, the intracranial pressure measuring device 100 may further include an external monitoring device 200.

According to various exemplary embodiments of the present invention, the external monitoring device 200 may include, for example, at least one of a smartphone, a tablet personal computer (PC), a mobile phone, a video phone, an electronic book reader, a desktop PC, a laptop PC, a netbook computer, a workstation, a server, a personal digital assistant (PDA), a portable multimedia player (PMP), an MP3 player, a mobile medical instrument, a camera, and a wearable device (e.g., smart glasses, a head-mounted device (HMD), electronic clothes, an electronic bracelet, an electronic necklace, an electronic appcessory, an electronic tattoo, a smart mirror, or a smart watch).

The external monitoring device 200 may receive intracranial pressure measurement data from the intracranial pressure measuring device 100. For example, the external monitoring device 200 and the intracranial pressure measuring device 100 may be connected through, but not limited to, the 3rd generation partnership project (3GPP) network, the long term evolution (LTE) network, the world interoperability for microwave access (WiMAX) network, the Internet, the local area network (LAN), the wireless local area network (Wireless LAN), the wide area network (WAN), the personal area network (PAN), the Bluetooth network, the near field communication (NFC) network, the satellite broadcast network, the analog broadcast network, the digital multimedia broadcasting (DMB) network, or the like. The external monitoring device 200 may be connected to the intracranial pressure measuring device 100 in a wired manner through a connection module such as a cable connector.

The intracranial pressure measuring device 100 measures the intracranial pressure based on the measured pressure in the cerebral blood vessel of the subject and may transmit a measurement result to the external monitoring device 200. Based on the intracranial pressure measurement data, the external monitoring device 200 may visualize the measurement result in respect to the intracranial pressure of the subject in various forms such as a graph and provide a user with the measurement result, but the present application is not limited thereto.

The sensor unit 110 may measure a pressure at a predetermined position in a cerebral blood vessel of a subject. In this case, the pressure may be referred to as, but not limited to, a pressure in blood, a blood pressure, or a pressure of blood. In addition, according to one exemplary embodiment of the present application, the cerebral blood vessel of the subject may be, but not limited to, a cerebral vein, a cerebral artery, or a blood vessel of a dura mater. As an example, the cerebral blood vessel of the subject may be any one of the blood vessels positioned in the brain in which the pressure may be measured. The sensor unit 110 may be a pressure monitoring micro sensor. The sensor unit 110 has a wire shape (or a flexible shape that may be movable upward, downward, leftward, and rightward). The sensor unit 110 may be an element capable of measuring a pressure at a middle portion or an end of the wire unit 120, and an optical element or a piezo element may be positioned. However, the shape of the sensor or the element is not limited to the above-mentioned shape and element.

According to one exemplary embodiment of the present application, based on and in accordance with various shapes of the intracranial pressure measuring device 100, the sensor unit 110 may be disposed at a predetermined position and may measure the pressure at the predetermined position in the cerebral blood vessel.

As an example, referring to FIG. 3, the sensor unit 110 may be disposed in one region inside a stent shape that has a cylindrical structure opened at both ends thereof and may be inserted into the cerebral blood vessel. The stent may be formed as a metal mesh. The stent may autonomously shrink and expand and may have flexibility. A material of the stent may be variously determined from materials such as stainless steel, titanium, cobalt, and chromium. The stent supports the cerebral blood vessel, such that the sensor unit 110, which is provided in one region inside the stent, may more accurately measure the pressure in the cerebral blood vessel.

The sensor unit 110 may be provided in one region inside the stent. The wire unit 120 may be positioned at one end of the stent to position the stent including the sensor unit 110 at the predetermined position in the cerebral blood vessel. The wire unit 120 (e.g., a thin wire) of the stent is inserted through the cerebral blood vessel and accesses the predetermined position in the cerebral blood vessel, and the pressure at the predetermined position in the cerebral blood vessel may be measured.

As the stent autonomously expands in the cerebral blood vessel, the sensor unit 110 may move to be close to an inner wall of the cerebral blood vessel. For example, the stent may be positioned in a shrunk shape in the cerebral blood vessel until the stent reaches the predetermined position in the cerebral blood vessel. The stent autonomously expands when the stent reaches the predetermined position in the cerebral blood vessel, such that the stent may move to be close to the inner wall of the cerebral blood vessel. As an example, the inner wall of the cerebral blood vessel may be a cerebral vein adjacent to a cerebral parenchyma 2. That is, the sensor unit 110 may be positioned on the inner wall of the cerebral vein adjacent to the cerebral parenchyma 2 instead of an inner wall of a cerebral blood vessel adjacent to a cranium 1.

The intracranial pressure measuring device 100 may include a marker 140 for identifying a position or a direction of the sensor unit 110. The marker 140 may be provided to determine whether the position or the direction of the sensor unit 110 is positioned in the cerebral vein adjacent to the cerebral parenchyma 2. The stent may autonomously expand when the position or the direction of the marker 140 is positioned in the cerebral vein adjacent to the cerebral parenchyma 2. An example of the marker may be a marker that does not allow a radioactive ray to penetrate therethrough, and the position or the direction of the marker may be identified from the outside through a radioactive ray device and an image device.

According to one exemplary embodiment of the present application, the wire unit 120 may be connected to one side of the stent having one region in which the sensor unit 110 is disposed. The wire unit 120 may position the sensor unit 110 at the predetermined position in the cerebral blood vessel of the subject. When the sensor unit 110 is positioned at the predetermined position in the cerebral blood vessel of the subject, the wire unit 120 may be separated from the stent by means of heat, electricity, or the like provided in a separating unit 115. The wire unit 120 may be separated from the stent through the separating unit 115 and may be withdrawn from the cerebral blood vessel.

In contrast, the wire unit 120 may not be separated from the stent and transmits the measurement result in respect to the pressure in the cerebral blood vessel to the intracranial pressure measuring unit 130 through a separate information transmitting line included therein.

In addition, according to one exemplary embodiment of the present application, referring to FIG. 4, the intracranial pressure measuring device 100 has coil units 151 and 152 and may measure the pressure in the cerebral blood vessel. Since the intracranial pressure measuring device 100 has the coil units, it is possible to obstruct a blood flow in the cerebral blood vessel and measure the intracranial pressure through a pressure in an artery among the cerebral blood vessels.

According to one exemplary embodiment of the present application, the first coil unit 151 may be positioned at one side in a direction in which the sensor unit is inserted into the subject. The second coil unit 152 may be positioned at one side in a direction opposite to the direction in which the sensor unit is inserted into the subject. The wire unit 120 may be connected to at least one of the first coil unit 151, the sensor unit 110, and the second coil unit 152.

The first coil unit 151 is a distal coil unit and may prevent the blood from flowing into a distal end side based on the sensor unit 110. That is, the first coil unit 151 may minimize the inflow of the blood. In addition, the second coil unit 152 is a proximal coil unit and may prevent the blood from flowing into a proximal end side based on the sensor unit 110 or minimize the inflow of the blood.

When the first coil unit 151 and the second coil unit 152 are positioned at predetermined positions in the blood vessel, the first coil unit 151 and the second coil unit 152 autonomously expand in the cerebral blood vessel to obstruct the blood flow toward the sensor unit 110. As an example, the first coil unit 151 and the second coil unit 152 are in a shrunk state while the first coil unit 151 and the second coil unit 152 are inserted into the cerebral blood vessel, and the first coil unit 151 and the second coil unit 152 autonomously expand when the first coil unit 151 and the second coil unit 152 reach the predetermined positions in the cerebral blood vessel, thereby obstructing the blood flow toward the sensor unit 110.

An example of an operation of the intracranial pressure measuring device 100 will be described with reference to FIG. 4. The first coil unit 151 according to the exemplary embodiment of the present application may block a distal portion of the artery among the cerebral blood vessels by using coil embolization and may position the inserted sensor unit 110 at the predetermined position in the artery among the cerebral blood vessels after the coil embolization. After the sensor unit 110 is positioned at the predetermined position in the artery among the cerebral blood vessels, the second coil unit 152 may block the blood vessel at the predetermined position by using the coil embolization using the second coil unit 152 in order to isolate the sensor unit 110 in the artery. In this way, the blood flow cannot reach the sensor unit 110 or a small amount of blood may reach the sensor unit 110.

At least one of the first coil unit 151, the sensor unit 110, and the second coil unit 152 may be connected to the wire unit 120. The wire unit 120 may position the first coil unit 151, the sensor unit 110, and the second coil unit 152 at the predetermined positions in the cerebral blood vessel. After at least one of the first coil unit 151, the sensor unit 110, and the second coil unit 152 is positioned in the blood vessel, the wire unit 120 may be separated from the separating unit 115 connected to the second coil unit 152. The wire unit 120 may be separated and withdrawn through the blood vessel of the subject. The wire unit 120 may be separated from at least one of the first coil unit 151, the sensor unit 110, and the second coil unit 152 by means of heat, electricity, or the like provided in the separating unit 115.

In addition, although not illustrated in FIG. 4, the intracranial pressure measuring device 100 may include the marker 140 for identifying the position or the direction of the sensor unit 110. The marker 140 may be provided to determine whether the position or the direction of the sensor unit 110 is positioned in the cerebral blood vessel adjacent to the cerebral parenchyma 2. The marker may be a marker that does not allow a radioactive ray to penetrate therethrough, and the position or the direction of the marker may be identified from the outside through the radioactive ray device and the image device.

According to one exemplary embodiment of the present application, the sensor unit 110 may include an antenna for transmitting the measurement result. The antenna may transmit the measurement result to the intracranial pressure measuring unit 130 in a wireless manner.

In addition, according to one exemplary embodiment of the present application, the sensor unit 110 and the wire unit 120 may be connected in a wired manner, and the pressure measurement result of the sensor unit 110 may be obtained. The separate information transmitting line is included in the wire unit 120 and may transmit the measurement result in respect to the pressure in the cerebral blood vessel. As another example, the information transmitting line may be positioned outside the wire unit and may be attached to the wire unit. In addition, the sensor unit 110 may be a wire type sensor. The wire type sensor is connected to an external electrode, and the intracranial pressure measuring device 100 may monitor the measurement result in respect to the pressure in the cerebral blood vessel.

In addition, according to one exemplary embodiment of the present application, a transmitter (not illustrated) may transmit the measurement result in respect to the pressure in the cerebral blood vessel. The transmitter (not illustrated) may be positioned in a blood vessel of an arm or a neck of a subject and may transmit the measurement result in respect to the pressure in the cerebral blood vessel to the intracranial pressure measuring unit 130.

The intracranial pressure measuring unit 130 may measure the intracranial pressure based on the pressure in the cerebral blood vessel which is measured by the sensor unit 110. When the intracranial pressure, which is a predetermined pressure or higher, is measured, the intracranial pressure measuring unit 130 may transmit a warning signal to the transmitter (not illustrated). In addition, the intracranial pressure measuring unit 130 may measure the intracranial pressure for each specific time (e.g., every 10 seconds) based on the pressure in the cerebral blood vessel which is measured by the sensor unit 110.

FIG. 5 is a view schematically illustrating an example of a process of measuring the intracranial pressure by applying the first exemplary embodiment of the intracranial pressure measuring device according to the exemplary embodiment of the present application. The process of measuring the intracranial pressure illustrated in FIG. 5 is performed by the intracranial pressure measuring device 100 described above with reference to FIGS. 1 to 4. Therefore, the contents described in respect to the intracranial pressure measuring device 100 with reference to FIGS. 1 to 4 are applied to FIG. 5 even though the contents are omitted.

Referring to FIG. 5, unlike the intracranial pressure measuring method in the related art which measures a pressure in the cerebral parenchyma by boring a hole in the cranium 1, the intracranial pressure measuring device 100 may measure the intracranial pressure by measuring the pressure in the cerebral blood vessel in a minimally invasive manner by applying the vascular access method used for angiography. The intracranial pressure measuring device 100 has the cylindrical structure which is opened at both ends thereof and has the stent shape, and the intracranial pressure measuring device 100 may be inserted into the cerebral blood vessel. The sensor unit 110 is disposed in one region in the stent, and based on the position or the direction in which the sensor unit 110 is disposed in the stent, the markers 151 and 152 may be disposed in the same region in which the sensor unit 110 is positioned in the stent. The stent in which the sensor unit 110 is disposed may be inserted, in an autonomously shrunk state, into the blood vessel until the stent reaches the predetermined position in the cerebral blood vessel. The stent in which the sensor unit 110 is disposed may be connected to the wire unit 120 in one region. The wire unit 120 is inserted into the subject and may position the sensor unit 110 at the predetermined position in the cerebral blood vessel. When the sensor unit 110 and the markers 151 and 152 are positioned on the inner wall of the cerebral blood vessel adjacent to the direction of the cerebral parenchyma 2, the stent, which reaches the predetermined position in the cerebral blood vessel, autonomously expands and may be positioned maximally close to the inner wall of the cerebral blood vessel. As another example, even though the intracranial pressure measuring device 100 does not have the markers 151 and 152, the sensor unit 110 may be positioned so that a sensor included in the sensor unit 110 is adjacent to the direction of the cerebral parenchyma 2.

The wire unit 120 may be separated, through the separating unit 115, from the stent in which the sensor unit 110 is disposed. The sensor unit 110 separated from the wire unit 120 is positioned in the cerebral blood vessel, thereby enabling the pressure in the cerebral blood vessel to be continuously monitored. To transmit the measurement result, the sensor unit 110 may transmit the intracranial pressure measurement result data to the intracranial pressure measuring unit 130 in a wireless manner through the antenna. In addition, as another example, the wire unit 120 includes the separate information transmitting line and may transmit the measurement result in respect to the pressure in the cerebral blood vessel measured by the sensor unit to the intracranial pressure measuring unit 130.

According to one exemplary embodiment of the present application, the intracranial pressure measuring device 100 may include the transmitter (not illustrated) for transmitting the measurement result in respect to the pressure in the cerebral blood vessel. The transmitter (not illustrated) may be positioned under the skin of the arm or the neck of the subject (e.g., an arm vein or a neck vein). For example, the measurement result in respect to the pressure in the cerebral blood vessel measured by the sensor unit 110 may be transmitted through the antenna, and the transmitter (not illustrated) may transmit the measurement result in respect to the pressure in the cerebral blood vessel to the intracranial pressure measuring unit 130. In addition, the transmitter (not illustrated) may transmit the measurement result in respect to the pressure in the cerebral blood vessel to the external monitoring device 200.

However, the exemplary embodiment described with reference to FIG. 5 is just one of the exemplary embodiments of the present invention, the present invention is not limited to the exemplary embodiment, and various exemplary embodiments may be present.

It will be appreciated that the exemplary embodiments of the present application have been described above for purposes of illustration, and those skilled in the art may understand that the present application may be easily modified in other specific forms without changing the technical spirit or the essential features of the present application. Therefore, it should be understood that the above-described exemplary embodiments are illustrative in all aspects and do not limit the present application. For example, each component described as a single type may be carried out in a distributed manner. Likewise, components described as a distributed type can be carried out in a combined type.

The scope of the present application is represented by the claims to be described below rather than the detailed description, and it should be interpreted that the meaning and scope of the claims and all the changes or modified forms derived from the equivalent concepts thereto fall within the scope of the present application.

What is claimed is:

1. An intracranial pressure measuring device comprising:
a sensor unit which measures a pressure at a predetermined position in a cerebral blood vessel of a subject;
a wire unit which is connected to the sensor unit and inserted into the subject to position the sensor unit at the position; and
an intracranial pressure measuring unit which measures an intracranial pressure based on the measured pressure.

2. The intracranial pressure measuring device of claim 1, further comprising:
a stent which has a cylindrical structure opened at both ends thereof, is inserted into the cerebral blood vessel, and has one region therein in which the sensor unit is disposed,
wherein the wire unit is connected to the stent.

3. The intracranial pressure measuring device of claim 2, wherein the sensor unit moves to be close to an inner wall of the cerebral blood vessel as the stent autonomously expands in the cerebral blood vessel.

4. The intracranial pressure measuring device of claim 3, wherein the sensor unit moves to be close to the inner wall in a direction of cerebral parenchyma.

5. The intracranial pressure measuring device of claim 4, further comprising:
a marker for identifying a position or a direction of the sensor unit.

6. The intracranial pressure measuring device of claim 5, wherein a position of the marker is determined based on a position or a direction in which the sensor unit is disposed in the stent.

7. The intracranial pressure measuring device of claim 2, wherein the cerebral blood vessel is a cerebral vein.

8. The intracranial pressure measuring device of claim 1, further comprising:
a first coil unit positioned at one side in a direction in which the sensor unit is inserted into the subject; and
a second coil unit positioned at one side in a direction opposite to the direction,
wherein the wire unit is connected to at least one of the first coil unit, the sensor unit, and the second coil unit.

9. The intracranial pressure measuring device of claim 8, wherein the first coil unit and the second coil unit obstruct a blood flow toward the sensor unit.

10. The intracranial pressure measuring device of claim 8, wherein the cerebral blood vessel is a cerebral artery.

11. The intracranial pressure measuring device of claim 9, wherein the first coil unit and the second coil unit obstruct a blood flow toward the sensor unit as the first coil unit and the second coil unit autonomously expand in the cerebral blood vessel.

12. The intracranial pressure measuring device of claim 1, wherein the sensor unit includes an antenna for transmitting a measurement result, the antenna transmits the measurement result to the intracranial pressure measuring unit in a wireless manner, and the intracranial pressure is measured based on the measurement result.

13. The intracranial pressure measuring device of claim 1, wherein the wire unit includes a separate information transmitting line in a wire and transmits a measurement result in respect to the pressure in the cerebral blood vessel measured by the sensor unit to the intracranial pressure measuring unit.

14. The intracranial pressure measuring device of claim 1, further comprising:
a transmitter for transmitting a measurement result in respect to the pressure in the cerebral blood vessel,
wherein the transmitter is positioned in a blood vessel of an arm or a neck of the subject and transmits the measurement result in respect to the pressure in the cerebral blood vessel to the intracranial pressure measuring unit.

15. The intracranial pressure measuring device of claim 1, wherein the wire unit positions the sensor unit at a predetermined position in the cerebral blood vessel of the subject, and the wire unit is separated and withdrawn from the cerebral blood vessel.
